(19) United States
(12) Patent Application Publication
(10) Pub. No.: US 2020/0377831 A1
(43) Pub. Date: Dec. 3, 2020

(54) SYSTEMS AND METHODS FOR PROCESSING NON-FERMENTED LIQUIDS

(71) Applicant: Pint At Home, LLC, Evergreen, CO (US)

(72) Inventors: James Kohler, Evergreen, CO (US); Michael Rissi, Downingtown, PA (US)

(73) Assignee: Pint At Home, LLC, Evergreen, CO (US)

(21) Appl. No.: 16/606,636

(22) PCT Filed: Apr. 19, 2018
(86) PCT No.: PCT/US2018/028306

§ 371 Date: Oct. 18, 2019

Related U.S. Application Data
(60) Provisional application No. 62/487,164, filed on Apr. 19, 2017.

(51) Int. Cl. C12C 11/07; C12C 7/28; C12G 3/024; C12C 13/02; C12N 1/16

(52) U.S. Cl. CPC C12C 11/075; C12C 7/285; C12N 1/16; C12C 13/02; C12G 3/024

(57) ABSTRACT

Embodiments described herein generally relate to systems and methods for processing non-fermented liquids. In some embodiments, non-fermented liquids may be processed into fermentable liquids in relatively short time (e.g., less than or equal to 30 minutes). In certain embodiments, the non-fermented liquids may be processed in relatively small batches (e.g., having a volume of less than or equal to 2 liters). The systems and methods described herein may be useful for producing fermented (e.g., alcoholic) beverages by a consumer. Advantageously, the systems and methods may be for use by a consumer where, upon introduction of a non-fermented liquid into the system, a fermented beverage is produced (e.g., in relatively small batches and/or in relative short times) as compared to traditional fermentation systems and methods (e.g., requiring relatively long fermentation times and/or relatively large batches). In certain embodiments, the systems and methods produce fermented beverages in a substantially continuous manner (e.g., as compared to traditional fermentation systems which utilize batch and/or semi-batch processes). Such systems may be useful for, for example, on-demand brewing of alcoholic beverages.

### RELATED APPLICATIONS

This application claims priority under 35 U.S.C. § 119(e) to U.S. Provisional Patent Application Ser. No. 62/487,164, filed Apr. 19, 2017, and entitled “Systems And Methods For Processing Non-Fermented Liquids,” which is incorporated herein by reference in its entirety for all purposes.

### FIELD OF THE INVENTION

The present invention generally relates to systems and methods for processing non-fermented liquids including, for example, fermenting the liquid.

### BACKGROUND

Fermented beverages such as beer and wine are often produced in large batches of relatively high volume which can be time consuming, expensive, and/or may involve the risk the loss of large quantities if the process in producing the fermented beverage is not properly executed. Furthermore, many factors may affect the taste, alcohol content, and other desirable properties of the fermented beverage that cannot be easily controlled during such large batch production. As such, improved systems and methods for producing fermented beverages are needed.

### SUMMARY OF THE INVENTION

The present invention provides systems and methods for processing non-fermented liquids including, for example, fermenting the liquid. The subject matter of the present invention involves, in some cases, interrelated products, alternative solutions to a particular problem, and/or a plurality of different uses of one or more systems and/or articles.

In one aspect, systems are provided. In some embodiments, the system comprises a fermentation device, configured to receive a non-fermented composition and to ferment at least 100 mL of the non-fermented composition into a fermented liquid having an alcoholic content of at least 4% in less than or equal to 30 minutes.

In another aspect, methods are provided. In some embodiments, the method comprises providing, to a fermentation device, a non-fermented composition, fermenting at least 100 mL of the non-fermented composition to have an alcoholic content of at least 4% in less than or equal to 30 minutes.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

FIGS. 1A-1B are schematic drawings illustrating a non-fermented component, according to one set of embodiments.

FIGS. 1C-1D are schematic drawings illustrating a fermenting component, according to one set of embodiments.

FIGS. 1E-1I are schematic illustrations of exemplary shapes of a fermenting component, according to one set of embodiments.

FIG. 1J is a schematic drawing illustrating a component of a system, according to one set of embodiments.

FIG. 1K is a schematic drawing illustrating a component of a system, according to one set of embodiments.

FIG. 2A is a schematic drawing illustrating a fermentation device comprising a fermenting component, according to one set of embodiments.

FIG. 2B is a schematic drawing illustrating a fermentation device comprising a fermenting component, according to one set of embodiments.

FIG. 3 is a schematic drawing illustrating a system for processing non-fermented compositions, according to one set of embodiments.

FIG. 4 is a schematic drawing illustrating a system for processing non-fermented compositions, according to one set of embodiments.

Other aspects, embodiments and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

Embodiments described herein generally relate to systems and methods for processing non-fermented liquids. In some embodiments, non-fermented liquids may be processed into fermentable liquids in relatively short time (e.g., less than or equal to 30 minutes). In certain embodiments, the non-fermented liquids may be processed in relatively small batches (e.g., having a volume of less than or equal to 2 liters). The systems and methods described herein may be useful for producing fermented (e.g., alcoholic) beverages by e.g., a consumer.

Advantageously, the systems and methods may be for use by a consumer where, upon introduction of a non-fermented liquid into the system, a fermented beverage is produced (e.g., in relatively small batches and/or in relative short times) as compared to traditional fermentation systems and methods (e.g., requiring relatively long fermentation times and/or relatively large batches). In certain embodiments, the systems and methods produce fermented beverages in a substantially continuous manner (e.g., as compared to traditional fermentation systems which utilize batch and/or semi-batch processes). Such systems may be useful for, for example, on-demand brewing of alcoholic beverages. In some embodiments, the systems and methods described herein may be relatively easy to use and may be e.g., easy for users to set up, operate, and/or clean as compared to traditional fermentation systems and methods.

In certain embodiments, the systems and methods described herein may generate alcoholic content in a relatively large volume of liquid (e.g., comprising a non-fermented composition) in a relatively short amount of time, as compared to traditional fermentation (e.g., brewing) methods. In an exemplary embodiment, the system is configured to receive a non-fermented composition and ferments at least 100 mL of the non-fermented composition into a fermented liquid having an alcoholic content of at least 1% (e.g., at least 4%) in less than or equal to 90 minutes (e.g., 30 minutes).

In some embodiments, the system comprises one or more compositions and/or one or more components (e.g., a fermenting device) such that a non-fermented composition interacts (e.g., contacts, directly contacts, reacts with) a fermenting composition, such that the non-fermented composition increases in alcoholic content (e.g., relative to the alcoholic content of the non-fermented composition prior to interacting with the fermenting composition). In some embodiments, the fermenting composition ferments at least a portion of the non-fermented composition.

Non-limiting examples of non-fermented compositions (e.g., a non-fermented liquids) suitable for use in the systems and methods described herein include drinkable liquids, wort (e.g., beer wort), carbonated liquids (e.g., soda), honey, juices (e.g., grape juice, apple juice) and malt. Non-limiting examples of fermented liquids that may be produced by the systems and methods described herein (e.g., from non-fermented liquids) include beer, mead, cider, wine, and other alcoholic beverages. In some cases, the fermented liquid is carbonated. The non-fermented composition may optionally include a variety of additives, such as sugar, electrolytes, caffeine, salt(s), flavoring, vitamins, herbs, amino acids, tea extracts, seed extracts, fruit extracts. The non-fermented composition may further include a variety of drinkable liquids, such as a fruit juice, coffee, tea, a sports drink, an energy drink, soda pop, milk, or the like.

In some embodiments, the non-fermented compositions described herein (e.g., the non-fermented liquids) may be provided to and/or present in the system in an encapsulated form (e.g., a spherical droplet). In an exemplary embodiment, the non-fermented composition (e.g., wort) may be encapsulated. Advantageously, encapsulation of the non-fermented composition may, for example, reduce the overall weight of the non-fermented composition needed, increase shelf-life (e.g., decrease rate of deterioration and/or perishability of the non-fermented composition), and/or increase the surface area of the non-fermented composition available to interact (e.g., react) with a fermenting composition such as yeast. Any suitable means may be used to encapsulate the non-fermented composition including, for example, a spherification process, droplet pipetting, straining meshes, microfluidic droplet generators, emulsification, or the like to form encapsulated non-fermented compositions. In an exemplary embodiment, the non-fermented compositions are encapsulated using a spherification process (e.g., a spherification process, a reverse spherification process). In an exemplary embodiment, sodium alginate and calcium chloride, pectin, agar, gelatin, pectin, and/or calcium glucate lactate may be reacted with the non-fermented compositions and formed into a shape such as a sphere, an ovoid, or the like. In some embodiments, the non-fermented composition may be encapsulated with a hydrogel.

In certain embodiments, the encapsulated non-fermented composition may be configured to release the non-fermented composition from the encapsulated state in the presence of applied heat (e.g., greater than or equal to 27° C.), upon chemical reaction of a reactant with the encapsulating material, and/or exposure to a liquid such as water, as described in more detail below.

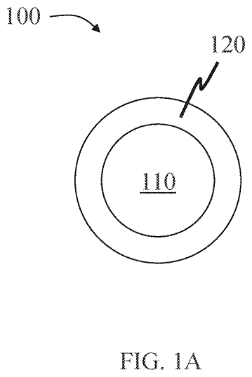

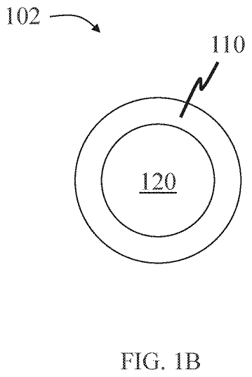

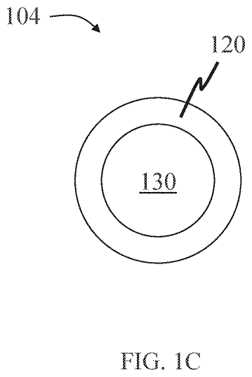

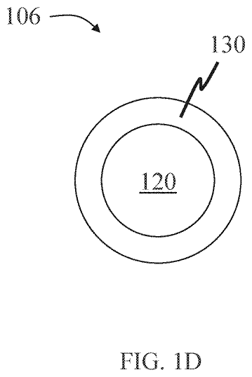

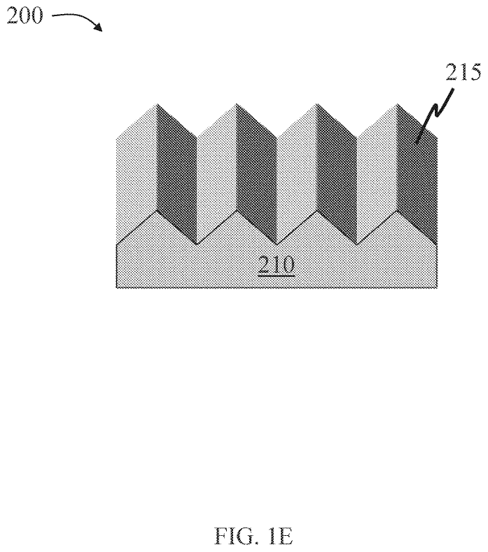

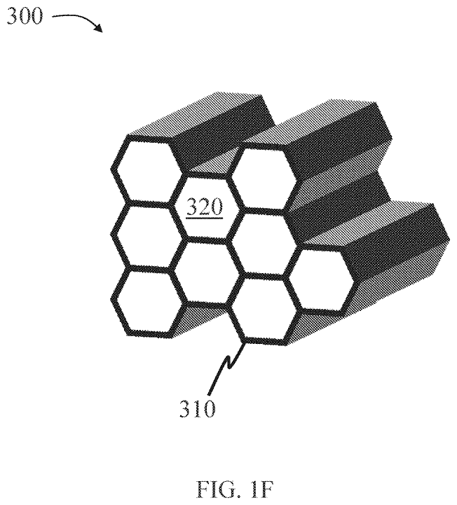

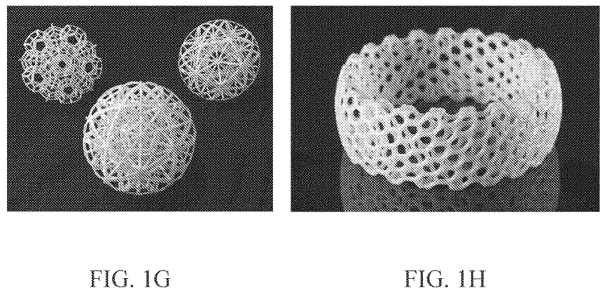

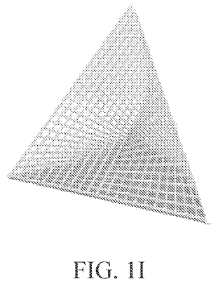

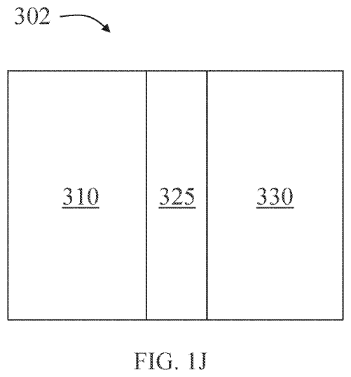

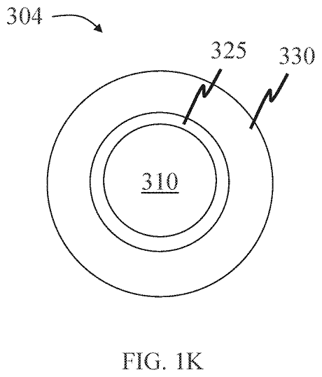

As illustrated in FIG. 1A, in some embodiments, a non-fermented component 100 (e.g., formed using spherification) comprises non-fermented composition 110 surrounded and/or encapsulated e.g., by material 120. In some cases, material 120 may be a semi-permeable membrane, as described in more detail below. In certain embodiments, material 120 comprises sodium alginate and calcium chloride, pectin, agar, gelatin, pectin, and/or calcium glucate lactate, as described above. In some embodiments, the non-fermented component is formed using a reverse spherification process. For example, as illustrated in FIG. 1B, in certain embodiments, non-fermented component 102 (e.g., formed using reverse spherification) comprises material 120 surrounded by and/or encapsulated by non-fermented composition 110. While FIGS. 1A and 1B illustrates spherical non-fermented components, one of ordinary skill in the art would understand that the non-fermented component may have any suitable shape including, for example, cylindrical, ovoid, prismatic, or the like. In certain embodiments, the non-fermented component may have an average cross-sectional dimension (e.g., diameter) of greater than or equal to 0.25 mm, greater than or equal to 0.5 mm, greater than or equal to 1 mm, greater than or equal to 2 mm, greater than or equal to 3 mm, greater than or equal to 5 mm, greater than or equal to 7 mm, greater than or equal to 10 mm, greater than or equal to 12 mm, greater than or equal to 15 mm, greater than or equal to 17 mm, greater than or equal to 20 mm, or greater than or equal to 22 mm. In some embodiments, the non-fermented component has an average cross-sectional dimension (e.g., diameter) of less than or equal to 25 mm, less than or equal to 22 mm, less than or equal to 17 mm, less than or equal to 15 mm, less than or equal to 12 mm, less than or equal to 10 mm, less than or equal to 7 mm, less than or equal to 5 mm, less than or equal to 3 mm, less than or equal to 2 mm, less than or equal to 1 mm, or less than or equal to 0.5 mm. Combinations of the above-referenced ranges are also possible (e.g., greater than or equal to 0.25 mm and less than or equal to 25 mm, greater than or equal to 0.25 mm and less than or equal to 10 mm). Other ranges are also possible.

The non-fermented component, in some cases, may be added to the system e.g., to ferment the non-fermented composition. For example, a user may, in some cases, add a non-fermented component comprising the non-fermented composition to the system, such that the non-fermented composition is at least partially fermented. In some embodiments, the non-fermented component is present in the system and remains unfermented, until exposed to the fermenting composition. In some embodiments, the non-fermented composition is provided in a kit.

In some cases, the non-fermented components may be provided to (or present in) the system as a plurality of encapsulated non-fermented compositions. For example, in certain embodiments, a plurality of encapsulated non-fermented compositions (e.g., spheres comprising an encapsulated non-fermented composition) may be arranged within the system (e.g., within an enclosure) e.g., such that the encapsulated non-fermented compositions are packed closely together. In some embodiments, the plurality of encapsulated non-fermented compositions are arranged within an enclosure and have a particular packing density. In certain embodiments, the packing density of the encapsulated non-fermented compositions are greater than or equal to 50%, greater than or equal to 55%, greater than or equal to 60%, greater than or equal to 64%, greater than or equal to 65%, greater than or equal to 70%, or greater than or equal to 72%. In some embodiments, the packing density of the encapsulated non-fermented compositions may be less than or equal to 74%, less than or equal to 72%, less than or equal to 70%, less than or equal to 65%, less than or equal to 64%, less than or equal to 60%, or less than or equal to 55%. Combinations of the above referenced ranges are also possible (e.g., greater than or equal to 50% and less than or equal to 74%). Other ranges are also possible. In some embodiments, at least a portion of the encapsulated non-fermented compositions may be arranged in any suitable arrangement including, for example, a cubic arrangement, a body-centered cubic arrangement, a face-centered cubic arrangement, a hexagonal close packed arrangement, or a random arrangement. Other arrangements are also possible.

In certain embodiments, the non-fermented composition may be encapsulated with a semi-permeable membrane. For example, in some cases, the encapsulating material is semi-permeable. Advantageously, the use of semi-permeable membranes to encapsulate the non-fermented composition may, for example, in some cases increase the fermentation rate of the non-fermented composition as compared to traditional methods (e.g., batch mixing of non-fermented compositions in solution). Without wishing to be bound by theory, as described above, the encapsulation of the non-fermented composition may increase the surface area available to interact (e.g., react) with a fermenting composition such as yeast. Any suitable material may be used to encapsulated the non-fermented composition including, for example, an alginate gel. Non-limiting examples of suitable encapsulating materials include calcium alginate, polyethylene terephthalate, calcium lactate, calcium lactate gluconate, sodium alginate, calcium salt, alginate baths, xanthan, agar, carrageenan, sodium pyrophosphate, and combinations or copolymers thereof.

In some embodiments, the encapsulated non-fermented composition may comprise the non-fermented composition and water. In certain embodiments, the amount of water present in the encapsulated non-fermented composition may be less than or equal to 30 vol %, less than or equal to 25 vol %, less than or equal to 20 vol %, less than or equal to 15 vol %, less than or equal to 10 vol %, less than or equal to 5 vol %, less than or equal to 4 vol %, less than or equal to 3 vol %, less than or equal to 2 vol %, or less than or equal to 1 vol % versus the total volume of the encapsulated non-fermented composition. In some embodiments, the amount of water present in the encapsulated non-fermented composition is greater than or equal to 0.1 vol %, greater than or equal to 1 vol %, greater than or equal to 2 vol %, greater than or equal to 3 vol %, greater than or equal to 4 vol %, greater than or equal to 5 vol %, greater than or equal to 10 vol %, greater than or equal to 15 vol %, greater than or equal to 20 vol %, or greater than or equal to 25 vol % versus the total volume of the encapsulated non-fermented composition. Combinations of the above-referenced ranges are also possible (e.g., less than or equal to 30 vol % and greater than or equal to 0.1 vol %, less than or equal to 5 vol % and greater than or equal to 0.1 vol %). Other ranges are also possible.

In some embodiments, the non-fermented composition (e.g., wort) is present in the non-fermented component in an amount greater than or equal to 20 wt %, greater than equal to 25 wt %, greater than equal to 30 wt %, greater than equal to 35 wt %, greater than equal to 40 wt %, greater than equal to 50 wt %, greater than equal to 60 wt %, greater than or equal to 70 wt %, greater than or equal to be weak percent, greater than or equal to 90 wt %, greater than equal to 95 wt %, or greater than equal to 98 wt % versus the total weight of the non-fermented component. In certain embodiments, the non-fermented composition may be present in the non-fermented component in an amount of less than or equal to 100 wt %, less than or equal to 98 wt %, less or equal to 95 wt %, less than equal to 98 wt %, less than or equal to 80 wt %, less than or equal to 70 wt %, less than or equal to 60 wt %, less than or equal to 50 wt %, less than equal to 40 wt %, less than equal to 35 wt %, less than equal to 30 wt %, or less than or equal to 25 wt %, versus the total weight of the non-fermented component. Combinations of the above referenced ranges are also possible (e.g., greater than equal to 20 wt %and less than or equal to 100 wt %, greater than equal to 50 wt %and less than or equal to 100 wt %). Other ranges are also possible. The non-fermented component, in some embodiments, may also comprise one or more additives such as polymers and/or binders e.g., comprising the remaining weight of the component. Non-limiting examples of suitable additives include calcium alginate, pet, calcium lactate, calcium lactate gluconate, sodium alginate, calcium salt, alginate baths, xanthan, agar, carrageenan, sodium pyrophosphate, sucrose solution, calcium chloride, and combinations thereof or copolymers thereof.

The non-fermented composition (e.g., the encapsulated non-fermented composition) may be provided to the system such that the non-fermented composition is at least partially fermented, as described above. In some embodiments, the non-fermented composition is released from the capsule before, during, and/or after fermentation. For example, the non-fermented composition may, in some cases, be at least partially fermented while encapsulated. In certain embodiments, the non-fermented composition is at least partially released (e.g., fully released) from the encapsulated form such that it may interact with a fermenting composition. The non-fermented composition may be released from the encapsulated form using any suitable means including, for example, dissolution of the encapsulating material, breaking of the encapsulating material, and/or bursting of the encapsulating material. In some cases, the non-fermented component may be heated and/or exposed to a fluid such as water such that the non-fermented composition is released.

For example, referring again to FIG. 1A, material 120 may, prior to and/or during exposure to the fermenting composition, dissolve or otherwise break such that non-fermented composition 110 is at least partially released from non-fermented component 100. In some cases, the non-fermented composition is fermented and then may be released from the capsule.

In some embodiments, the system ferments at least 100 mL (e.g., at least 140 mL, at least 170 mL, at least 200 mL, at least 250 mL, at least 473 mL, at least 500 mL, at least 1000 mL, at least 1065 mL, at least 1863 mL, at least 3549 mL) of a non-fermented composition to a percent alcoholic content of at least 1 vol % (e.g., at least 4 vol %, at least 5 vol %, at least 10 vol %, at least 15 vol %) in less than or equal to 90 minutes, less than or equal to 75 minutes, less than or equal to 60 minutes, less than or equal to 45 minutes, 30 minutes, less than or equal to 25 minutes, less than or equal to 20 minutes, less than or equal to 15 minutes, less than or equal to 10 minutes, less than or equal to 8 minutes, less than or equal to 5 minutes, or less than or equal to 3 minutes. In certain embodiments, the system ferments at least 100 mL (e.g., at least 140 mL, at least 170 mL, at least 200 mL, at least 250 mL, at least 473 mL, at least 500 mL, at least 1000 mL, at least 1065 mL, at least 1863 mL, at least 3549 mL) of a non-fermented composition to a percent alcoholic content of at least 1 vol % (e.g., at least 4 vol %, at least 5 vol %, at least 10 vol %, at least 15 vol %) in greater than or equal to 1 minute, greater than or equal to 2 minutes, greater than or equal to 3 minutes, greater than or equal to 5 minutes, greater than or equal to 8 minutes, greater than or equal to 10 minutes, greater than or equal to 15 minutes, greater than or equal to 20 minutes, greater than or equal to 25 minutes, greater than or equal to 30 minutes, greater than or equal to 45 minutes, greater than or equal to 60 minutes, or greater than or equal to 75 minutes. Combinations of the above-referenced ranges are also possible (e.g., less than or equal to 30 minutes and greater than or equal to 5 minutes, less than or equal to 90 minutes and greater than or equal to 1 minute). Other ranges are also possible. Those of ordinary skill in the art would understand that fermentation to a percent alcoholic content of at least 4 vol % is not intended to include the addition of alcohol to a solution, but refers to the fermentation of one or more materials within the non-fermented composition (e.g., sugar) such that at least a portion of the material is (chemically) converted to alcohol (e.g., ethanol).

While much of the description above is related to fermentation of non-fermented compositions, one of ordinary skill in the art would understand based upon the teachings of this specification that fermentation of already at least partially fermented compositions is also possible. For example, in some embodiments, an at least partially fermented composition (e.g., having a non-zero alcohol content) may be added to the systems described herein and fermented such that the alcoholic content of the composition increases. In some such embodiments, the system increases the alcoholic content (e.g., ethanol) of at least 100 mL (e.g., at least 140 mL, at least 170 mL, at least 200 mL, at least 250 mL, at least 473 mL, at least 500 mL, at least 1000 mL, at least 1065 mL, at least 1863 mL, at least 3549 mL) by at least 1 vol % (e.g., at least 4 vol %, at least 5 vol %, at least 10 vol %, at least 15 vol %) in less than or equal to 90 minutes, less than or equal to 75 minutes, less than or equal to 60 minutes, less than or equal to 45 minutes, 30 minutes, less than or equal to 25 minutes, less than or equal to 20 minutes, less than or equal to 15 minutes, less than or equal to 10 minutes, less than or equal to 8 minutes, less than or equal to 5 minutes, or less than or equal to 3 minutes.

In certain embodiments, the system increases the alcoholic content of at least 100 mL (e.g., at least 140 mL, at least 170 mL, at least 200 mL, at least 250 mL, at least 473 mL, at least 500 mL, at least 1000 mL, at least 1065 mL, at least 1863 mL, at least 3549 mL) by at least 1 vol % (e.g., at least 4 vol %, at least 5 vol %, at least 10 vol %, at least 15 vol %) in greater than or equal to 2 minutes, greater than or equal to 3 minutes, greater than or equal to 5 minutes, greater than or equal to 8 minutes, greater than or equal to 10 minutes, greater than or equal to 15 minutes, greater than or equal to 20 minutes, greater than or equal to 25 minutes, greater than or equal to 30 minutes, greater than or equal to 45 minutes, greater than or equal to 60 minutes, or greater than or equal to 75 minutes. Combinations of the above-referenced ranges are also possible (e.g., less than or equal to 30 minutes and greater than or equal to 5 minutes, less than or equal to 90 minutes and greater than or equal to 3 minutes). Other ranges are also possible.

In certain embodiments, the system ferments the non-fermented composition to a percent alcoholic content (ethanol) of at least 1 vol % (e.g., at least 4 vol %, at least 5 vol %, at least 10 vol %, at least 15 vol %) at a rate of greater than or equal to 10 mL/min, greater than or equal to 20 mL/min, greater than or equal to 30 mL/min, greater than or equal to 40 mL/min, greater than or equal to 50 mL/min, greater than or equal to 75 mL/min, greater than or equal to 100 mL/min, greater than or equal to 125 mL/min, greater than or equal to 150 mL/min, greater than or equal to 175 mL/min, greater than or equal to 200 mL/min, greater than or equal to 250 mL/min, or greater than or equal to 350 mL/min. In some embodiments, the system ferments the non-fermented composition to a percent alcoholic content of greater than or equal to 1 vol % (e.g., at least 4 vol %, at least 5 vol %, at least 10 vol %, at least 15 vol %) at a rate of less than or equal to 500 mL/min, less than or equal to 350 mL/min, less than or equal to 250 mL/min, less than or equal to 200 mL/min, less than or equal to 175 mL/min, less than or equal to 150 mL/min, less than or equal to 125 mL/min, less than or equal to 100 mL/min, less than or equal to 75 mL/min, less than or equal to 50 mL/min, less than or equal to 40 mL/min, less than or equal to 30 mL/min, or less than or equal to 20 mL/min. Combinations of the above-referenced ranges are also possible (e.g., greater than or equal to 10 mL/min and less than or equal to 200 mL/min, greater than or equal to 10 mL/min and less than or equal to 500 mL/min). Other ranges are also possible.

In some embodiments, the system increases the alcoholic (ethanol) content of a composition by greater than or equal to 1 vol % (e.g., at least 4 vol %, at least 5 vol %, at least 10 vol %, at least 15 vol %) at a rate of greater than or equal to 10 mL/min, greater than or equal to 20 mL/min, greater than or equal to 30 mL/min, greater than or equal to 40 mL/min, greater than or equal to 50 mL/min, greater than or equal to 75 mL/min, greater than or equal to 100 mL/min, greater than or equal to 125 mL/min, greater than or equal to 150 mL/min, or greater than or equal to 175 mL/min, greater than or equal to 200 mL/min, greater than or equal to 250 mL/min, or greater than or equal to 350 mL/min. In some embodiments, the system increases the alcoholic (ethanol) content of a composition by greater than or equal to 1 vol % (e.g., at least 4 vol %, at least 5 vol %, at least 10 vol %, at least 15 vol %) at a rate of less than or equal to 500 mL/min, less than or equal to 350 mL/min, less than or equal to 250 mL/min, less than or equal to 200 mL/min, less than or equal to 175 mL/min, less than or equal to 150 mL/min, less than or equal to 125 mL/min, less than or equal to 100 mL/min, less than or equal to 75 mL/min, less than or equal to 50 mL/min, less than or equal to 40 mL/min, less than or equal to 30 mL/min, or less than or equal to 20 mL/min. Combinations of the above-referenced ranges are also possible (e.g., greater than or equal to 10 mL/min and less than or equal to 200 mL/min). Other ranges are also possible. In certain embodiments, the system ferments (e.g., to a percent alcoholic content of at least 1 vol %, by an increase of at least 1 vol %, to a percent alcoholic content of at least 4 vol %, by an increase of at least 4 vol %) a volume of at least 100 mL, at least 250 mL, at least 500 mL, at least 1 L, or at least 1.5 L of a non-fermented composition. In some embodiments, the system is configured to ferment (e.g., to a percent alcoholic content of at least 1 vol %, by an increase of at least 1 vol %, to a percent alcoholic content of at least 4 vol %, by an increase of at least 4 vol %) a volume of less than or equal to 2 L, less than or equal to 1.5 L, less than or equal to 1 L, less than or equal to 500 mL, or less than or equal to 250 mL. Combinations of the above-referenced ranges are also possible (e.g., at least 100 mL and less than or equal to 4 L). Other ranges are also possible. In some embodiments, the fermentation of the non-fermented composition occurs continuously (e.g., at least a portion of the non-fermented composition is under continuous fluidic flow during fermentation).

Fermentation of the non-fermented composition may be characterized, in some embodiments, by the alcohol (ethanol) concentration generated during fermentation. In some cases, the system ferments a non-fermented composition (e.g., having a volume of at least 100 mL) to an alcohol concentration of at least 1 vol %, at least 2 vol %, at least 4 vol %, at least 5 vol %, at least 7 vol %, least 9 vol %, at least 10 vol %, at least 12 vol %, at least 15 vol %, at least 20 vol %, or at least 25 vol % versus the total volume of composition. In certain embodiments, the system ferments a non-fermented composition (e.g., having a volume of at least 100 mL) to an alcohol concentration of less than or equal to 30 vol %, less than or equal to 25 vol %, less than or equal to 20 vol %, less than or equal to 15 vol %, less than or equal to 12 vol %, less than or equal to 10 vol %, less than or equal to 9 vol %, less than or equal to 7 vol %, less than or equal to 5 vol %, less than or equal to 4 vol %, or less than or equal to 2 vol % versus the total volume of composition. Combinations of the above-referenced ranges are also possible (e.g., at least 1 vol % and less than or equal to 30 vol %, at least 4 vol % and less than or equal to 30 vol %). Other ranges are also possible. Alcohol (ethanol, concentration as described herein may be determined using high-performance liquid chromatography (HPLC) on 100 mL of solution at 20° C.

In certain embodiments, the system increases the alcohol (ethanol) content of a composition (e.g., at least partially fermented and having a volume of at least 100 mL) by at least 1 vol %, at least 2 vol %, at least 4 vol %, at least 5 vol %, at least 7 vol %, least 9 vol %, at least 10 vol %, at least 12 vol %, at least 15 vol %, at least 20 vol %, or at least 25 vol % versus the total volume of composition. In certain embodiments, the system increases the alcohol (ethanol) content of a composition (e.g., at least partially fermented and having a volume of at least 100 mL) by less than or equal to 30 vol %, less than or equal to 25 vol %, less than or equal to 20 vol %, less than or equal to 15 vol %, less than or equal to 12 vol %, less than or equal to 10 vol %, less than or equal to 9 vol %, less than or equal to 7 vol %, less than or equal to 5 vol %, less than or equal to 4 vol %, or less than or equal to 2 vol % versus the total volume of composition. Combinations of the above-referenced ranges are also possible (e.g., at least 1 vol % and less than or equal to 30 vol %, at least 4 vol % and less than or equal to 30 vol %). Other ranges are also possible. Alcohol (ethanol, concentration as described herein may be determined using high-performance liquid chromatography (HPLC) on 100 mL of solution at 20° C.

In some embodiments, the system comprises a fermentation device (e.g., a fermentation chamber). In certain embodiments, the fermentation device is configured to receive the non-fermented composition and/or the non-fermented component (e.g., comprising the encapsulated non-fermented composition) such that it may be fermented. In some cases, the fermentation device may comprise and/or be configured to receive a fermenting composition (e.g., yeast such as immobilized yeast). In some embodiments, a fermenting component comprises the fermenting composition.

In certain embodiments, the fermenting component may have a spherical shape and/or a non-spherical shape. In some cases, the fermenting component may have a shape selected to increase the surface area to volume ratio (e.g., as compared to a flat layer, as compared to a sphere). Without wishing to be bound by theory, increasing the surface area to volume ratio may advantageously increase the surface area at which fermentation takes place such that fermentation rates increase as compared to traditional fermentation methods. In some cases, the fermenting composition (e.g., yeast) may be encapsulated as described above in the context of the non-fermented composition. For example, referring now to FIG. 1C, in some embodiments, fermenting component 104 comprises fermenting composition 130 at least partially encapsulated by material 120 (e.g., formed by a spherification process). In some embodiments, as illustrated in FIG. 1D, material 120 is at least partially encapsulated by fermenting composition 130 (e.g., formed by a reverse spherification process).

In some embodiments, the fermenting component may have a spherical shape. In certain embodiments, the fermenting component may be spherical and have an average cross-sectional dimension of greater than or equal to 0.25 mm, greater than or equal to 0.5 mm, greater than or equal to 1 mm, greater than or equal to 2 mm, greater than or equal to 3 mm, greater than or equal to 5 mm, greater than or equal to 7 mm, greater than or equal to 10 mm, greater than or equal to 12 mm, greater than or equal to 15 mm, or greater than or equal to 20 mm. In some embodiments, the fermenting component has a spherical shape and has an average cross-sectional dimension of less than or equal to 25 mm, less than or equal to 20 mm, less than or equal to 15 mm, less than or equal to 12 mm, less than or equal to 10 mm, less than or equal to 7 mm, less than or equal to 5 mm, less than or equal to 3 mm, less than or equal to 2 mm, less than or equal to 1 mm, or less than or equal to 0.5 mm. Combinations of the above-referenced ranges are also possible (e.g., greater than or equal to 0.25 mm and less than or equal to 25 mm). Other ranges are also possible.

In certain embodiments, the fermenting component may have a cylindrical shape such as a tube or coiled shape (e.g., such as a spring). For example, in some cases, the fermentation component may have a substantially cylindrical shape having a particular average diameter along the length of the shape. In some cases, the fermenting component has cylindrical shape having an average diameter of greater than or equal to 0.1 mm, greater than or equal to 0.3 mm, greater than or equal to 0.5 mm, greater than or equal to 1 mm, greater than or equal to 2 mm, greater than or equal to 3 mm, greater than or equal to 5 mm, greater than or equal to 7 mm, greater than or equal to 10 mm, greater than or equal to 12 mm, greater than or equal to 15 mm, or greater than or equal to 17 mm. In certain embodiments, the fermenting component has a cylindrical shape having an average diameter of less than or equal to 20 mm, less than or equal to 17 mm, less than or equal to 15 mm, less than or equal to 12 mm, less than or equal to 10 mm, less than or equal to 7 mm, less than or equal to 5 mm, less than or equal to 3 mm, less than or equal to 2 mm, less than or equal to 1 mm, less than or equal to 0.5 mm, or less than or equal to 0.3 mm. Combinations of the above-referenced ranges are also possible (e.g., greater than or equal to 0.1 mm and less than or equal to 20 mm). Other ranges are also possible.

As described above, the fermenting component may be configured such that it comprises a relatively high surface area to volume ratio. For example, the fermenting component may comprise a plurality of ridges, grooves, roughness, and/or other surface features and/or textures such that the surface area of the fermenting component is increased relative to a relatively flat surface. In some embodiments, as illustrated in FIG. 1E, fermenting component 200 comprises layer 210 (e.g., comprising a fermenting composition), layer 210 comprising a grooved surface 215. Layer 210, in some embodiments, may comprise a fermenting composition as described herein and one more additional materials (e.g., calcium alginate, polyethylene terephthalate, calcium lactate, calcium lactate gluconate, sodium alginate, calcium salt, alginate baths, xanthan, agar, carrageenan, sodium pyrophosphate, and combinations or copolymers thereof). For example, as described above in the context of spherification, layer 210 may comprise the fermenting composition encapsulated by another material (e.g., sodium alginate, polymers, etc.). That is to say, in some cases, surface 215 may comprise the other material with fermenting composition contained therein. In some embodiments, the fermenting composition is not encapsulated, and is embedded within layer 210 (e.g., embedded within a matrix in layer 210). The surface features (e.g., ridges, groups, texture) of a surface of the fermenting component may have any suitable a largest average cross-sectional dimension (e.g., height). For example, in some embodiments, the largest average cross-sectional dimension (e.g., height) of the surface features of the fermenting component may be greater than equal to 0.1 microns, greater than equal to 0.5 microns, greater than equal to 1 microns, greater than equal to 2 microns, greater than equal to 5 microns, greater than equal to 10 microns, greater than or equal to 20 microns, greater than equal to 50 microns, greater than equal to hundred microns, greater than equal to 200 microns, or greater than equal to 500 microns. In some embodiments, the largest average cross-sectional dimension of the surface features of the fermenting component may be less than or equal to 750 microns, less than or equal to 500 microns, less than equal to 200 microns, less than or equal to 100 microns, less than equal to 50 microns, less than or equal to 20 microns, less than or equal to 10 microns, less than or equal to 5 microns, less than or equal to 2 microns, less than or equal to 1 microns, or less than or equal to 0.5 microns. Combinations of the above-referenced ranges are also possible (e.g., greater than equal to 0.1 microns and less than or equal to 750 microns). Other ranges are also possible. The surface features and/or textures may also have any suitable shape. Those of ordinary skill in the art would understand, based upon the teachings of this specification, how to select sizes and shapes for the surface features and/or textures. As described above, surface features and/or textures described herein may increase surface area of the fermenting component relative to a flat surface and, without wishing to be bound by theory, increase the rate of fermentation of a non-fermented composition exposed to the fermenting component.

In certain embodiments, the fermenting component may comprise a plurality of pores. In an exemplary embodiment, the fermenting component may comprise a honeycomb type structure. For example, as illustrated in FIG. 1F, fermenting component 300 comprises fermenting material 310 (e.g., comprising the fermenting composition) and a plurality of pores 320. In some embodiments, a non-fermented composition may be introduced into pores 320 such that the non-fermented composition is fermented by the fermenting composition within fermenting material 310. The course of the fermenting component may have any suitable cross-sectional size, length, and/or cross-sectional shape. For example, in some embodiments, the pores have a cross-sectional shape that is circular, oval, elliptical, square, polygonal (e.g., hexagonal), triangular, rectangular, trapezoidal. and/or irregular.

In some embodiments, the average cross-sectional dimension (e.g., diameter) of the pores of the fermenting component are greater than equal to 0.1 microns, greater than equal to 0.5 microns, greater than equal to 1 microns, greater than equal to 2 microns, greater than equal to 5 microns, greater than equal to 10 microns, greater than or equal to 20 microns, greater than equal to 50 microns, greater than equal to hundred microns, greater than equal to 200 microns, or greater than equal to 500 microns. In some embodiments, the average cross-sectional dimension of the pores of the fermenting component may be less than or equal to 750 microns, less than or equal to 500 microns, less than equal to 200 microns, less than or equal to 100 microns, less than equal to 50 microns, less than or equal to 20 microns, less than or equal to 10 microns, less than or equal to 5 microns, less than or equal to 2 microns, less than or equal to 1 microns, or less than or equal to 0.5 microns. Combinations of the above-referenced ranges are also possible (e.g., greater than equal to 0.1 microns and less than or equal to 750 microns). Other ranges are also possible. As described above, the pores described herein may increase the surface area of the fermenting component available to the non-fermented composition and, without wishing to be bound by theory, may increase the rate of fermentation of a non-fermented composition exposed to the fermenting composition.

In some cases, the fermenting component may be characterized by a particular surface area to volume ratio. That is to say, in some embodiments, the surface area of the fermenting component e.g., configured to contact the non-fermented component, relative to the volume of the fermenting component, is relatively high. Those of ordinary skill in the art would understand that the volume of the fermenting component does not refer to the total space occupied by the fermenting component as a whole (e.g., a convex hull of the fermenting component and/or defined by the largest cross-sectional dimension of the fermenting component) by, by contrast, refers to the volume occupied by the fermenting component material. For example, referring again to FIG. 1F, the volume of the non-fermented component comprises the volume occupied by fermenting material 310 and does not include the volume occupied by pores 320.

In certain embodiments, the fermenting component may have any suitable configuration and/or shape. For example, in some embodiments, the fermenting component may have a complex course structure such as illustrated in FIGS. 1G-1I. Non-limiting examples of suitable shapes for the fermenting component include, in some embodiments, porous cubes, porous tubes, porous cylinders, menger sponge, sierpinski tetrahedron, hilbert cube, and others.

In some cases, the fermenting component may comprise a plurality of encapsulated fermenting composition spheres (or ovoids) arranged within one or more shapes outlined above. In some embodiments, the plurality of encapsulated fermenting compositions may be arranged as described above in the context of the encapsulated non-fermented composition/component (e.g., arranged and/or having a particular packing density e.g., greater than or equal to 50% and less than or equal to 74% as described above).

The fermenting component may have any suitable exposed surface area (e.g., such that the non-fermented composition contacts at least a portion of the surface area of the fermenting component). In some embodiments, the surface area of the fermenting component may be greater than or equal to 5 cm², greater than or equal to 10 cm², greater than or equal to 20 cm², greater than or equal to 50 cm², greater than or equal to 100 cm², greater than or equal to 200 cm², greater than or equal to 500 cm², greater than or equal to 1000 cm², greater than or equal to 2000 cm², greater than or equal to 5000 cm², greater than or equal to 10,000 cm², greater than or equal to 20,000 cm², greater than or equal to 25,000 cm², or greater than or equal to 50,000 cm². In certain embodiments, the surface area of the fermenting component (for contact with the non-fermented composition) is less than or equal to 100,000 cm², less than or equal to 50,000 cm², less than or equal to 25,000 cm², less than or equal to 20,000 cm², less than or equal to 10,000 cm², less than or equal to 5000 cm², less than or equal to 2000 cm², less than or equal to 1000 cm², less than or equal to 500 cm², less than or equal to 200 cm², less than or equal to 100 cm², less than or equal to 50 cm², less than or equal to 20 cm², or less than or equal to 10 cm². Combinations of the above-referenced ranges are also possible (e.g., greater than or equal to 5 cm² and less than or equal to 100,000 cm², greater than or equal to 100 cm² and less than or equal to 25,000 cm²). Other ranges are also possible.

The fermenting component (and/or the non-fermented component) may be formed using any suitable means including, for example, 3-D printing, molding, spherification, and/or combinations thereof. In an exemplary embodiment, the fermenting component is formed using 3-D printing of a material comprising the fermenting composition (e.g., yeast). In another exemplary embodiment, the fermenting component is formed by a molding process. In yet another exemplary embodiment, the fermenting component is formed into a substantial planar honeycomb-type structure (e.g., comprising the fermenting composition and a plurality of pores), and rolled into a tube or roll.

In some embodiments, the fermenting composition (e.g., yeast) is present in the fermenting component in an amount greater than or equal to 20 wt %, greater than equal to 25 wt %, greater than equal to 30 wt %, greater than equal to 35 wt %, greater than equal to 40 wt %, greater than equal to 50 wt %, greater than equal to 60 wt %, greater than or equal to 70 wt %, greater than or equal to be weak percent, greater than or equal to 90 wt %, greater than equal to 95 wt %, or greater than equal to 98 wt % versus the total weight of the fermenting component. In certain embodiments, the fermenting composition may be present in the fermenting component in an amount of less than or equal to 100 wt %, less than or equal to 98 wt %, less or equal to 95 wt %, less than equal to 98 wt %, less than or equal to 80 wt %, less than or equal to 70 wt %, less than or equal to 60 wt %, less than or equal to 50 wt %, less than equal to 40 wt %, less than equal to 35 wt %, less than equal to 30 wt %, or less than or equal to 25 wt %, versus the total weight of the fermenting component. Combinations of the above referenced ranges are also possible (e.g., greater than equal to 20 wt %and less than or equal to 100 wt %, greater than equal to 50 wt %and less than or equal to 100 wt %). Other ranges are also possible. The fermenting component, in some embodiments, may also comprise one or more additives such as polymers and/or binders e.g., comprising the remaining weight of the component. Non-limiting examples of suitable additives include calcium alginate, pet, calcium lactate, calcium lactate gluconate, sodium alginate, calcium salt, alginate baths, xanthan, agar, carrageenan, sodium pyrophosphate, sucrose solution, calcium chloride, and combinations thereof or copolymers thereof.

In some embodiments, the fermenting composition is substantially dehydrated (e.g., dehydrated yeast). In an exemplary set of embodiments, the fermenting composition comprises immobilized yeast. In certain embodiments, the fermenting composition is at least partially hydrated. For example, in some embodiments, the amount of water present in the fermenting composition may be less than or equal to 30 vol %, less than or equal to 25 vol %, less than or equal to 20 vol %, less than or equal to 15 vol %, less than or equal to 10 vol %, less than or equal to 5 vol %, less than or equal to 4 vol %, less than or equal to 3 vol %, less than or equal to 2 vol %, or less than or equal to 1 vol % versus the total volume of the encapsulated non-fermented composition. In some embodiments, the amount of water present in the fermenting composition is greater than or equal to 0.1 vol %, greater than or equal to 1 vol %, greater than or equal to 2 vol %, greater than or equal to 3 vol %, greater than or equal to 4 vol %, greater than or equal to 5 vol %, greater than or equal to 10 vol %, greater than or equal to 15 vol %, greater than or equal to 20 vol %, or greater than or equal to 25 vol % versus the total volume of the encapsulated non-fermented composition. Combinations of the above-referenced ranges are also possible (e.g., less than or equal to 30 vol % and greater than or equal to 0.1 vol %, less than or equal to 5 vol % and greater than or equal to 0.1 vol %). Other ranges are also possible.

In certain embodiments, the fermenting composition (e.g., comprising yeast) may be pre-activated. Those of ordinary skill in the art would understand how to pre-activate fermenting compositions such as yeast for the systems described herein based upon the teachings of this specification including, for example, introducing water and/or sugar to the yeast.

Non-limiting examples of suitable species of yeast include Saccharomyces cerevisiae, Saccharomyces boulardi, Saccharomyces pastorianus, Saccharomyces bayanus, Brettanomyces Lambicus, Torulaspora delbrueckii, and Saccharomyces uvarum. Other species are also possible. Those of ordinary skill in the art would be capable of selecting suitable species of yeast based upon the type of beverage desired and the teachings of this specification.

In certain embodiments, the system comprises a component (e.g., a cartridge) comprising a fermentation portion (e.g., comprising the fermenting composition) and a non-fermented portion (e.g., comprising the non-fermented composition) adjacent the fermentation portion. As used herein, when a portion is referred to as being “adjacent” another portion, it can be directly adjacent to (e.g., in contact with) the portion, or one or more intervening components (e.g., a material) also may be present. A portion that is “directly adjacent” another portion means that no intervening component(s) is present. For example, as illustrated in FIG. 1J, in some embodiments, component 302 comprises fermentation portion 310 adjacent non-fermented portion 330. In some embodiments, material 325 may be disposed between fermentation portion 310 and non-fermented portion 330. Material 325 may comprise any suitable material (e.g., a semi-permeable membrane) including, for example, calcium alginate, polyethylene terephthalate, calcium lactate, calcium lactate gluconate, sodium alginate, calcium salt, alginate baths, xanthan, agar, carrageenan, sodium pyrophosphate, and combinations or copolymers thereof In some embodiments, a fermentation portion (e.g., comprising the fermenting composition) may at least partially encapsulate the non-fermented portion (e.g., comprising the non-fermented composition). For example, as illustrated in FIG. 1K, component 304 comprises fermentation portion 310 encapsulated by material 325 and further encapsulated by non-fermented portion 330. In some embodiments, the component comprising the fermentation portion and the non-fermented portion may be introduced (e.g., inserted) into the system and a fluid (e.g., water) added to the component such that the fluid contacts (e.g., circulates between) the fermentation portion and the non-fermented portion (e.g., such that the non-fermented composition may be at least partially fermented). Other configurations are also possible. For example, while FIG. 1K illustrates fermentation portion 310 encapsulated by non-fermented portion 330, those of ordinary skill in the art would understand based upon the teachings of this specification that fermentation portion may, in certain embodiments, encapsulate non-fermented portion.

While FIGS. 1J and 1K illustrate material 325 disposed between fermentation portion 310 and non-fermented portion 330, in some embodiments, material 325 may not be present and/or may be positioned in other configurations. For example, in some embodiments, fermentation portion 310 may be directly adjacent non-fermented portion 330 (i.e. no intervening layers may be present)

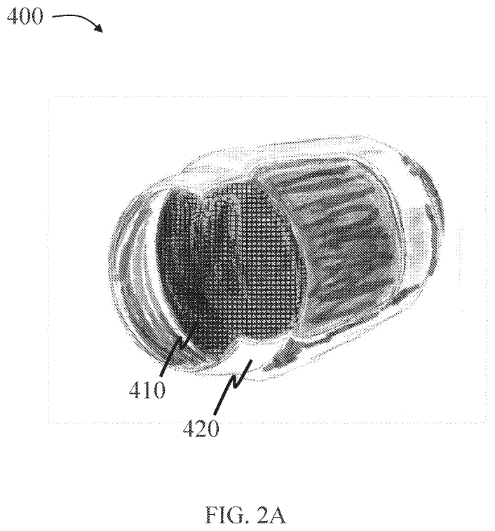

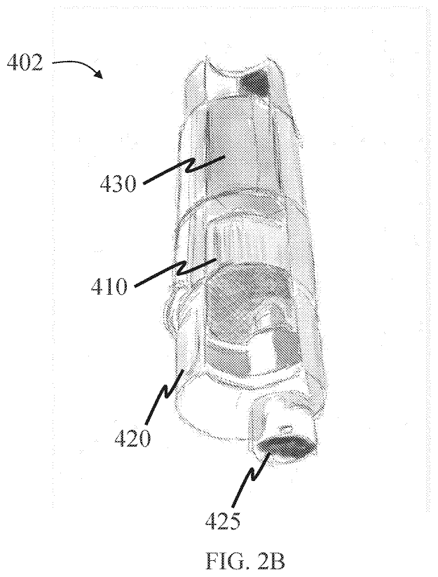

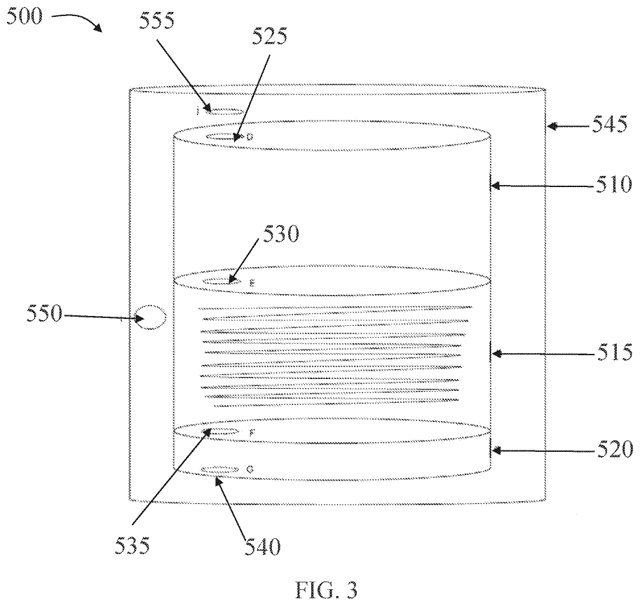

In some embodiments, the system comprises a fermentation device (e.g., a cartridge). In some cases, the fermentation device comprises the fermentation component and one or more inlets. For example, as illustrated in FIG. 2A, exemplary fermentation device 400 (e.g., a cartridge) comprises fermentation component 410 disposed within housing 420. As illustrated in FIG. 2B, in some embodiments, exemplary fermentation device 402 comprises fermentation component 410 disposed within housing 420 having an inlet 425. In some embodiments, inlet 425 is in fluidic communication with fermentation component 410 and/or non-fermented component 430. The fermentation device may be in fluidic communication with one or more additional components within the system including e.g., vessels, fluidic connectors, chambers, etc.

In some embodiments, fermentation component 410 and non-fermented component 430 may be in fluidic communication. For example, as illustrated in FIG. 2B, fermentation component 410 and non-fermented component 430 are in fluidic communication. In certain embodiments, the fermenting composition and/or the non-fermented composition may be in fluidic communication. In some cases, a fluid (e.g., water) may be introduced into the fermentation device such that the fluid contacts the fermenting composition and/or the non-fermented composition. In some such embodiments, the non-fermented composition may be fermented by the fermenting composition. In some embodiments, fermentation component 410 and non-fermented component 430 may be constructed and arranged adjacent to one another (e.g., as described above in the context of the fermentation portion and the non-fermented portion).

In an exemplary set of embodiments, the system (and/or the fermentation device) comprises a non-fermented component comprising an encapsulated non-fermented composition fluidically isolated from a fermentation component. Upon introduction of a fluid to the system (and/or fermentation device), the fluid releases the non-fermented composition from encapsulation such that the non-fermented composition is suspended in the fluid. In some embodiments, the fermenting component may be placed in fluidic communication with the non-fermented composition suspended in the fluid. For example, a membrane (e.g., a semi-permeable membrane) disposed between the non-fermented composition and the fermenting composition may be opened (e.g., dissolved, opened, physically removed) such that the fluid comprising the non-fermented composition contacts the fermenting composition (e.g., such that the fermenting composition at least partially ferments the non-fermented composition). The membrane may comprise any suitable material including, for example, calcium alginate, polyethylene terephthalate, calcium lactate, calcium lactate gluconate, sodium alginate, calcium salt, alginate baths, xanthan, agar, carrageenan, sodium pyrophosphate, and combinations or copolymers thereof. Other materials are also possible.

In certain embodiments, the system comprises a circulation component such that a fluid may be introduced and mixed with the non-fermented composition and fermenting composition as described above.

In some embodiments, a fluid may be introduced to the fermentation component and/or the fermenting composition such that the fermenting composition may be pre-activated. Upon pre-activation of the fermenting composition, the pre-activated fermenting composition may be placed in fluidic communication with and/or introduced to the non-fermented composition (e.g., such that the fermenting composition at least partially ferments the non-fermented composition).

In certain embodiments, the fermentation device comprises a plurality of encapsulated fermenting components (e.g., comprising the fermenting composition) as described above and herein. For example, encapsulated fermenting composition spheres (or otherwise) may be disposed within a housing of the fermentation device. In other embodiments, the fermenting component is porous, as described above.

In some embodiments, one or more components of the system (e.g., the fermentation device, the fermentation component, the non-fermented component) is at least partially biodegradable. In certain embodiments, one or more components and/or compositions (e.g., the fermentation component, the fermenting composition, the non-fermented component, the non-fermented composition), of the system is edible (i.e. non-toxic).

The term “toxic” refers to a substance showing detrimental, deleterious, harmful, or otherwise negative effects on a subject, tissue, or cell when or after administering the substance to the subject or contacting the tissue or cell with the substance, compared to the subject, tissue, or cell prior to administering the substance to the subject or contacting the tissue or cell with the substance. In certain embodiments, the effect is death or destruction of the subject, tissue, or cell. In certain embodiments, the effect is a detrimental effect on the metabolism of the subject, tissue, or cell. In certain embodiments, a toxic substance is a substance that has a median lethal dose (LD50) of not more than 500 milligrams per kilogram of body weight when administered orally to an albino rat weighing between 200 and 300 grams, inclusive. In certain embodiments, a toxic substance is a substance that has an LD50 of not more than 1,000 milligrams per kilogram of body weight when administered by continuous contact for 24 hours (or less if death occurs within 24 hours) with the bare skin of an albino rabbit weighing between two and three kilograms, inclusive. In certain embodiments, a toxic substance is a substance that has an LC50 in air of not more than 2,000 parts per million by volume of gas or vapor, or not more than 20 milligrams per liter of mist, fume, or dust, when administered by continuous inhalation for one hour (or less if death occurs within one hour) to an albino rat weighing between 200 and 300 grams, inclusive.

The term “non-toxic” refers to a substance that is not toxic. Toxic reagents include, e.g., oxidative stressors, nitrosative stressors, proteasome inhibitors, inhibitors of mitochondrial function, ionophores, inhibitors of vacuolar ATPases, inducers of endoplasmic reticulum (ER) stress, and inhibitors of endoplasmic reticulum associated degradation (ERAD). In some embodiments a toxic reagent selectively causes damage to nervous system tissue. Toxic reagents include compounds that are directly toxic and reagents that are metabolized to or give rise to substances that are directly toxic. It will be understood that the term “toxic compounds” typically refers to reagents that are not ordinarily present in a cell's normal environment at sufficient levels to exert detectable damaging effects. However, in some cases, the toxic reagents may be present in a cell's normal environment but at concentrations significantly less than present in the auxiliary materials described herein. Typically toxic reagents exert damaging effects when present at a relatively low concentration, e.g., at or below 1 mM, e.g., at or below 500 microM, e.g., at or below 100 microM. It will be understood that a toxic reagents typically has a threshold concentration below which it does not exert detectable damaging effects. The particular threshold concentration will vary depending on the agent and, potentially, other factors such as cell type, other agents present in the environment, etc.

In certain embodiments, the fermentation device comprises one or more microfluidic components (e.g., channels, vessels). The term ‘microfluidic component’ generally refers to a component having an internal volume of less than 1000 microliters and greater than or equal to 1 microliter. In one set of embodiments, a semi-permeable membrane is in fluidic communication with a least one microfluidic channel of the fermentation device. In some such embodiments, the flow of a fluid through the semi-permeable membrane induces laminar flow of the fluid. Advantageously, the fermentation device comprising one or more microfluidic components may continuously process (e.g., ferment) relatively low volumes of fluid at relative high rates (e.g., fermenting in relatively short amounts of time). In some embodiments, the system may be configured to receive one or more cartridges. In certain embodiments, a cartridge comprises the non-fermented component and/or composition. In some cases, a cartridge comprises a fermenting composition. For example, a user may insert a cartridge comprising the non-fermented composition and/or the fermenting composition such that, within the system, the non-fermented composition is fermented as described herein.

In some embodiments, the cartridge comprises a semi-permeable hydrofluidic membrane. In certain embodiments, the cartridge comprises one or more strains of yeast (e.g., for fermentation of one or more materials present in the non-fermented composition, such as sugar) associated with, and separated by, the semi-permeable membrane. For example, the cartridge may comprise a first (micro)fluidic channel and a second (micro)fluidic channel in fluidic communication with the first (micro)fluidic channel. In some such cases, the semi-permeable membrane may be disposed between the first and second (micro)fluidic channels.

In some embodiments, the cartridge may be disposable (e.g., the cartridge may be a one-time use cartridge for fermenting a non-fermentable composition). In certain embodiments, the cartridge may be reusable (e.g., the cartridge may be washed and/or refilled with one or more strains of yeast between uses). Advantageously, the use of such cartridges (e.g., one-time use, reusable) may reduce and/or eliminate contamination during the fermentation process.

One or more components of the system described herein may also be reusable.

In certain embodiments, (e.g., upon insertion of the cartridge into the fermentation device) at least a portion of the non-fermented composition may be flowed across the semi-permeable membrane such that the non-fermented composition interacts with the one or more strains of yeast. In some cases, the flow of the non-fermented composition across the semi-permeable membrane may be laminar. During flow of the non-fermented composition, the composition may become fermented and/or carbonated.

In certain embodiments, the fermentation device comprises one or more vessels. In some embodiments, the vessel is configured to receive a non-fermented composition (e.g., a non-fermented liquid). In some embodiments, the non-fermented composition is semi-liquid (e.g., comprising one or more types of solid materials suspended in a liquid). In some cases, the non-fermented liquid may comprise water. In some embodiments, one or more vessels of the fermentation device may be used for storing a non-fermented composition and/or a fermented liquid. In some cases, one or more materials and/or liquids within a vessel may be mixed (e.g., by agitation such as via compressed carbon dioxide).

In some cases, one or more components, channels, and/or vessels of the may be sealed (e.g., with a breakable seal). In some embodiments, the seal may be broken such that one or more fluids and/or compositions flow. For example, in an exemplary embodiment, one or more channels (and/or vessels) are sealed and, upon inserted of the cartridge into the fermentation device, the seal is broken such that the non-fermented composition flows.

In some embodiments, the fermentation device comprises a water treatment component. For example, a vessel of the fermentation device may be used to perform water treatment to increase the quality of water for use in the system. In some embodiments, the water treatment component is configured to perform water calibration. For example, in certain embodiments, calibration may comprise reseting the liquid to zero impurities through filtration and or distillation. Once reset, the water treatment chamber may, in some cases, re-add major chemicals or minerals including but not limited to calcium, magnesium, sodium, carbonate bicarbonate, sulfite and chloride as well as potassium, iron, copper, zinc and ammonia. In some embodiments, the water treatment component may reduce or eliminate chloramine (e.g., which is toxic to yeast).

In certain embodiments, the system may comprise a rehydration, mixing, and cooling (RMC) chamber. In some embodiments, the RMC chamber is in fluidic communication with the water treatment component and configured to receive treated water (or other liquids). In certain embodiments, the RMC chamber is configured to receive a non-fermented component. In some cases, the RMC chamber may be configured to mix a non-fermented composition with treated water (or other liquids). For example, the RMC chamber may spin the mixture comprising the non-fermented composition and treated water at relatively high velocities (e.g., to achieve homogenous mixing). In some embodiments, the RMC chamber may be heated to a temperature of greater than or equal to 18° C., greater than or equal to 20° C., greater than or equal to 25° C., greater than or equal to 30° C., greater than or equal to 35° C., greater than or equal to 40° C., or greater than or equal to 43° C. For example, in some embodiments, the non-fermented component may comprise the non-fermented composition and an encapsulating layer such that, upon heating to a temperature of greater than or equal to 18° C., the non-fermented composition is released from the non-fermented component.

Advantageously, heating the RMC chamber may also be useful for pre-activating the fermenting composition (e.g., the yeast).

In some cases, the RMC chamber may be used to mix at least partially fermented liquids with additional fermenting components/compositions, such that the alcohol content of the liquid increases (e.g., continuously).

In certain embodiments, the RMC chamber may cool the fermented mixture. Non-limiting examples of suitable means for cooling the fermented mixture include compressed carbon dioxide, electronic cooling elements, centripetal chilling, and the like.

In certain embodiments, the fermentation device comprises one or more sensors (e.g., for measuring and/or determining the quality of the fermented liquid produced by the device).

The sensors may be selected from the group consisting of a carbon dioxide sensor, a photometer sensor, a spectrometer, a moisture sensor, a flow rate sensor, a specific gravity sensor, a temperature sensor, and combinations thereof. In an exemplary embodiment, at least one sensor may provide a profile of the produced beverage/fermented liquid. For example, the sensor may provide characteristics of the fermented liquid such as specific gravity, ethanol content, pH, color, carbon dioxide, serving temperature, amino acids, clarity, and/or bitterness.

In some cases, the fermentation device comprises one or more fluidic pumps (e.g., for managing the flow rate of the non-fermented composition through one or more components of the fermentation device such as the materials (e.g., the encapsulating materials described herein, the semi-permeable materials such as the semi-permeable membrane described herein).

One or more components of the fermentation device may be aesthetically pleasing. For example, the system may be designed to have an aesthetically pleasing design for consumer use (e.g., at home, at a restaurant, at a business, at an event). In some embodiments, the fermentation device, or one or more components therein, may have any suitable shape. For example, in certain embodiments, the fermentation device has a cylindrical shape, a cubic shape, a cuboidal shape, a prismatic shape, or a conical shape. In some cases, at least one cross-section of the components and/or the fermentation device may be rectangular shaped, square shaped, triangular shaped, circular shaped, hexagonal shaped, or irregularly shaped. Other shapes are also possible. In an exemplary embodiment, the fermentation device has a cylindrical shape.

The fermentation device may have also a base that, in some cases, is aesthetically pleasing. In some embodiments, the base comprises one or more electronic components (e.g., a microprocessor) in electrical communication with one or more components (e.g., a sensor) of the fermentation device. In some cases, the fermentation device and/or the base comprises a display (e.g., an LED screen) which may be used for, for example, providing a user (e.g., consumer) information such as but not limited to progress (i.e. status) of the fermentation process.

In some embodiments, one or more components of the fermentation device comprises a controller and/or microprocessor. In certain embodiments, the controller is configured (e.g., programmed) to receive and transmit data commands to/from one or more components of the fermentation device and/or the base. In some embodiments, the data includes one or more signals from one or more sensors. In some embodiments, the controller may be configured to adjust various parameters based on external metrics. For example, in certain embodiments, the controller is configured adjust the flow rate, fermentation rate, and/or target alcohol concentration in response to a signal from a sensor in electrical communication with the controller. In some embodiments, the controller adjusts the flow rate, fermentation rate, and/or target alcohol concentration in response to an input from the user and/or a signal from the sensor.

In some embodiments, the controller may include one or more proportional, integral, and/or derivative (PID) feedforward and/or feedback loops to adjust the flow rate, fermentation rate, and/or target alcohol concentration (e.g., in response to one or more sensors in communication with the controller). The controller may be implemented by any suitable type of analog and/or digital circuitry. For example, the controller may be implemented using hardware or a combination of hardware and software. When implemented using software, suitable software code can be executed on any suitable processor (e.g., a microprocessor) or collection of processors. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, or other tangible, non-transitory computer-readable storage medium) encoded with a computer program (i.e., a plurality of executable instructions) that, when executed on one or more processors, performs the above-discussed functions of one or more embodiments. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

In some cases, the microprocessor may be used to determine various parameters of the fermentation process and quality including, for example, specific gravity, density, alcohol by volume, temperature, carbonation output, carbonation input, ambient air pressure, moisture content, and/or attenuation of light (e.g., to calculate both fermentation progress and quality). In some embodiments, such parameters may be provided by one or more sensors of the system.

In some cases, the fermentation device and/or one or more components therein may be insulated (e.g., thermally insulated).

In some embodiments, a packaging device is provided. For example, the packaging device may be useful for preparing a non-fermented composition (e.g., for use in the systems and methods described herein). For example, in some cases, the packaging device may comprise one or more components for at least partially dehydrating at least a portion of the non-fermented composition. In some such cases, the at least partially dehydrated non-fermented composition may be provided to a consumer for fermentation in the system described herein. In certain embodiments, the packing device encapsulates the non-fermented composition as described here. Non-limiting methods for encapsulating the non-fermented composition include, for example, spherification, reverse spherification, automated pipetting, straining mesh, and combinations thereof.

In certain embodiments, the packaging device comprises a mechanical interface configured to receive a non-fermented composition (e.g., a non-fermented liquid). In some cases, the packaging device may comprise a pre-processing storage chamber configured for non-fermented compositions. In some embodiments, a dehydration chamber may be in fluidic communication with the storage chamber and is configured to reduce the concentration of H₂O in the non-fermented composition.

In some embodiments, the packaging device comprises a bath chamber (e.g., comprising agar) configured to encapsulate non-fermented compositions into capsules (e.g., gelatin capsules, agar capsules, etc.), as described herein. In some cases, a desired H₂O profile (e.g., concentration) may be reproduced with a gelling solution (e.g., agar solution).

In certain embodiments, the system comprises a self-cleaning (e.g., an automatic self-cleaning component) and/or sanitation component. In an exemplary embodiment, the automatic self-cleaning component is configured to use distilled H₂O at about 100° C. (e.g., for cleaning one or more components of the system).

### EXAMPLES

The following examples illustrate embodiments of certain aspects of the invention. It should be understood that the methods and/or materials described herein may be modified and/or scaled, as known to those of ordinary skill in the art.

### Example 1

The following example describes the formation of encapsulated non-fermented compositions, according to some embodiments described herein. Wort is created in a traditional wort brewing process. However, prior to transferring the wort to a fermentation tank, the wort is reduced to an extract by removing water. The reduction ratio of water to wort is generally between 50-95%. Once reduced, a specification process to encapsulate the wort so that we reduce the its weight for shipping efficiency and decrease deterioration or perishability.
a. The specification solidifies using the following criteria;
   i. Temperature of solidification 32 degrees celsius (89.6 F).
   ii. A 2% ratio between the weight of wort to dry agar used. For example if 2 grams agar would be used with 200 ml or wort.
b. The spheres are generally created using an automated pipette machine or a straining mesh such that the precise amount of wort is encapsulated.
c. The wort spheres are then bathed in calcium chloride to form a gelled alginate skin which is permeable to smaller molecules, conducive to fermentation. The spheres are then packaged inside wort chamber with potentially, small amount of H2O, wort and or the water minerals outlined in Water Treatment above #1.

### Example 2

The following example describes the formation of a fermenting component, according to some embodiments described herein.

To create the mixture, sodium alginate powder at a ratio of 3.25% may be used e.g., if yeast was 10 g and water 100 g (110 g*3.25%) or 3.75g of sodium alginate. Distilled water was mixed with sodium alginate at 160 degrees F., dissolved and run through a vacuum press to remove all air. The yeast may be then chilled to room temperature to form a yeast mixture.

The yeast mixture may then be loaded into a pressured canister, which is used to push the yeast mixture into a mold. Liquid yeast is used in the process.

Once the yeast mixture is injected into the mold, the mold may be submitted into a calcium chloride bath to gelatinize the yeast mixture into a 3D shape. The yeast mixture may also be deposited using 3D printing methods including additive manufacturing techniques.

The bath may be a mixture of 3.5% calcium chloride, and may be homogenously mixed e.g., such that absolutely no lumping occurs. The mold may then be placed into the bath, were the yeast mixture turns gelatinous as the calcium replaces sodium. The mold may then be washed in a bath of distilled water for sterilization. Upon completion, the yeast shape is generally porous with a rubber ball like texture and hollow which allows the flow of a non-fermented composition such as wort in and around the structure, enabling rapid fermentation and creation of carbon dioxide in the fermentation device.

### Example 3

The following example and Table 1 summarize the expected average rate of alcohol (ethanol) generation by fermentation of non-fermented compositions in an exemplary case having a 5.3 cm² surface area, according to some embodiments described herein.

TABLE 1

| | | | Time | mL |
|---|---|---|---|---|
| | Oz | mL | Min. | Per Min. |
| Pony | 7 | 207 | 3 | 76.67 |
| Pint | 16 | 473 | 6 | 78.86 |
| Growler 32 | 32 | 946 | 12 | 78.86 |
| Growler 64 | 64 | 1893 | 24 | 78.86 |
| Growler 128 | 128 | 3785 | 48 | 78.86 |

### Prophetic Example 1

The following example describes a prophetic example of systems, methods, and related components/devices for fermenting a non-fermented composition.

1. In some cases, a fermentation device is provided comprising: a thermally insulated cylindrical body shaped in visually pleasing manner; a internal vessel which contains water and non-fermented semi-liquid material; a secondary vessel which contains fermented liquids; a tertiary vessel inclusive a interchangeable cartridge used to perform water quality treatment; a visually pleasing base which interlocks with the thermally insulated cylindrical body allowing electronic communication; a carbon dioxide sensor, photometer sensor, a spectrometer, moisture sensor, flow rate sensor, specific gravity sensor and temperature sensor used for measuring the quality of the fermented output; a hydro-fluidic pump used for managing the flow rate of the non-fermented semi-liquid material through a interchangeable microfluidic membrane; a chamber which contains either non-fermented semi-liquid material or semi-liquid fermented material and uses compressed carbon dioxide to create a agitated mixing process with H₂O from the tertiary vessel.

2. A interchangeable microfluidic cartridge according to #1 wherein the cartridge contains a semi-permeable hydrofluidic membrane with one or more yeast strains and a method for forced carbonation, allowing the passage of non-fermented semi-liquid material across the membrane to interact with the yeast, using laminar flow to precisely control the flow of liquid through the microchannels and carbonate the liquid as it exits to the secondary vessel.

3. A base comprising: a cylindrical body shaped in a visually pleasing manner; a interlocking design with the fermentation device that allows communication of temperature, flow rate, carbon dioxide and specific gravity to a microprocessor; a ambient temperature sensor; a led display used to provide user information such as but not limited to the fermentation progress; a thermal induction magnet which interlocks with the fermentation device; a water calibration mechanism.

4. A microprocessor according to #3 wherein the microprocessor is programmed to receive data and transmit commands from and between the fermentation device and the base, including data from the temperature sensor, specific gravity sensor, flow rate sensor, carbon dioxide sensor and ambient pressure sensor to calculate fermentation process and quality.

5. A algorithmic method to calculate fermentation process and quality according to #3 wherein a algorithm uses specific gravity, density, alcohol by volume, temperature, carbonation output, carbonation input, ambient air pressure, moisture content, and attenuation of light to calculate both fermentation progress and quality.

6. A packaging device comprising, a mechanical interface to receive non-fermented liquid such as grape juice or beer wort, a pre-processing storage chamber for non-fermented liquids, a centrifugal dehydration chamber which receives non-fermented liquid and reduces the H₂O from the non-fermented liquid, a agar bath chamber which encapsulates the non-fermented liquid into gelatin capsules, a microprocessor which controls flow rates, water chemistry profile, centrifugal speed and communication with a cloud-computing system.

7. A packaging methodology according to #6 wherein a specific H₂O profile is reproduced with a agar solution, a algorithmic method to determine H₂O reduction, a algorithmic method to determine the quantity and size of gelatin capsules per e.g., 29.75 mL.

8. A water calibration mechanism according to #3 wherein a chamber contains user provided non-distilled H₂O that is then converted to distilled H₂O using vapor distillation. The water calibration mechanism interacts with the fermentation vessel and conducts agitated mixing of distilled H₂O with agar packaging which contains a specific water profile.

9. A self cleaning and sanitization methodology, automated self cleaning process using distilled H₂O heated to e.g., 100 degrees celsius.

10. One or more of the fermentation devices may be configured to permit remote interaction between the user and the device (e.g., via a wireless device). For example, a user can communicate with a device using a computer or other portable electronic device (e.g., a smartphone or tablet). In a particular example, a webpage or app can be configured to receive user input and communication the input to the device. The input may be, in some cases, related to the operation of the device and/or one or more desired settings (e.g., volume, alcohol content). In some cases, the user may receive information from the device (e.g., regarding the progress of the fermentation process). Such communication systems may occur through the Internet, a central server, and/or a cloud-computing system. In an exemplary case, the central server and/or cloud-computing system may be configured to collect and/or receive data from the fermentation device.

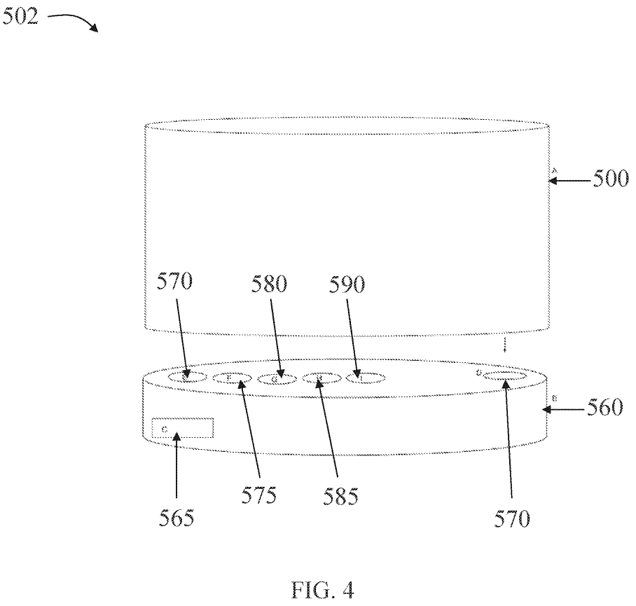

FIG. 3 shows an exemplary fermentation device 500 for fermenting a non-fermented composition. The exemplary fermentation device comprises:
a. Non-Fermented Liquid Chamber 510
b. Fermenting Component 515
c. CO2 Chamber 520
d. One-way Valve 525
e. Optional breakable seal 530
f. CO2 Entry Valve 535
g. Thermal Chamber Valve 540
h. Thermal Chamber 545
i. Optional Specific Gravity Sensor 550
j. Fluidic Pump 555

FIG. 4 shows an exemplary system 502 for integrating with the fermentation device 500 of FIG. 3. The exemplary system comprises:
a. Fermentation Device 500 (e.g., as illustrated in FIG. 3).
b. Base 560
c. MicroProcessor 565
d. Interlocking Communication 570
e. Optional Carbon Dioxide 575
f. Optional Photometer 580
g. Optional Spectrometer 585
h. Optional Moisture Sensor 590
i. Optional Temperature Sensor 595

The components in FIG. 3 and FIG. 4 are an exemplary embodiment and are not intended to be limiting. One of ordinary skill in the art would understand that not all of the components shown in the figures necessarily need be present in the system and/or one or more additional components not shown in the figures may be included (e.g., one or more additional sensors, channels, fluidic components, electrical components, etc.).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to.

Only the transitional phrases “consisting of” and “consisting essentially of” shall be closed or semi-closed transitional phrases, respectively, as set forth in the United States Patent Office Manual of Patent Examining Procedures, Section 2111.03.

Any terms as used herein related to shape and/or geometric relationship of or between, for example, one or more articles, structures, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape and/or geometric relationship include, but are not limited to terms descriptive of: shape—such as, round, square, circular/circle, rectangular/rectangle, triangular/triangle, cylindrical/cylinder, elliptical/ellipse, (n)polygonal/(n)polygon, etc.;

surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “ square,” as defined mathematically, to an extent typically achievable and achieved for the recited fabrication technique as would be understood by those skilled in the art or as specifically described.

1-39. (canceled)

40. A system, comprising:
a fermentation device, configured to receive a non-fermented composition and to ferment at least 100 mL of the non-fermented composition into a fermented liquid having an alcoholic content of at least 4% in less than or equal to 30 minutes.

41. A method, comprising:
providing, to a fermentation device, a non-fermented composition;
fermenting at least 100 mL of the non-fermented composition to have an alcoholic content of at least 4% in less than or equal to 30 minutes.

42. A method, comprising:
providing a fermenting component comprising a fermenting composition and a plurality of pores;
introducing, into the plurality of pores, a non-fermented composition such that the fermenting composition ferments the non-fermented composition to an alcoholic content of greater than or equal to 1 vol % at a rate of greater than or equal to 10 mL/min.

43. A system as in claim 40, wherein the fermentation device comprises one or more microfluidic components.

44. A system as in claim 40, wherein the fermentation device comprises a semi-permeable membrane.

45. A system as in claim 40, wherein the system is configured to receive a cartridge.

46. A system as in claim 44, wherein the cartridge comprises a semi-permeable membrane.

47. A system as in claim 44, wherein the cartridge comprises one or more strains of yeast.

48. A method as in claim 41, wherein fermenting comprises introducing H₂O and/or carbonation into the non-fermented composition.

49. A system as in claim 40, wherein the non-fermented composition is selected from the group consisting of drinkable liquids, wort, carbonated liquids, drinkable liquids, honey, juices and malt.

50. A system as in claim 40, wherein the non-fermented composition is encapsulated by an encapsulating material.

51. A system as in claim 50, wherein the encapsulating material comprises calcium alginate, polyethylene terephthalate, calcium lactate, calcium lactate gluconate, sodium alginate, calcium salt, alginate baths, xanthan, agar, carrageenan, sodium pyrophosphate, and combinations or copolymers thereof.

52. A system as in claim 50, wherein the encapsulating material is semi-permeable.

53. A system as in claim 40, wherein the encapsulated non-fermented composition has water present in an amount of less than or equal to 30 vol % and greater than or equal to 0.1 vol %.

54. A method as in claim 41, wherein at least 100 mL of the non-fermented composition is fermented to a percent alcoholic content of at least 1 vol % in less than or equal to 90 minutes.

55. A method as in claim 41, wherein the non-fermented composition is fermented to an alcoholic content of at least 1 vol % at a rate of greater than or equal to 10 mL/min and less than or equal to 500 mL/min.

56. A method as in claim 41, wherein the non-fermented composition is fermented to an alcoholic content of at least 1 vol % and less than or equal to 30 vol %.

57. A method as in claim 42, wherein the fermenting component comprises a plurality of ridges and/or grooves.

58. A method as in claim 42, wherein the fermenting component comprises surface features having a largest average cross-sectional dimension of greater than or equal to 0.1 microns and less than or equal to 750 microns.

59. A method as in claim 42, wherein the fermenting component comprises a plurality of pores.

60. A method as in claim 59, wherein the plurality of pores have an average cross-sectional dimension of greater than or equal to 0.1 microns and less than or equal to 750 microns.

61. A method as in claim 42, wherein a surface area of the fermenting component is greater than or equal to 5 cm² and less than or equal to 25,000 cm².

62. A method as in claim 42, wherein the fermenting composition is present in the fermenting component in an amount of greater than or equal to 20 wt % and less than or equal to 100 wt %.

63. A system as in claim 40, wherein the fermenting composition comprises yeast.

64. A system as in claim 40, wherein the fermenting component is adjacent the non-fermented composition.

65. A system as in claim 45, wherein the fermenting component and/or the non-fermented component are disposed within the cartridge.

66. A system as in claim 40, wherein the fermenting component is in fluidic communication with the non-fermented component.

67. A method as in claim 42, wherein the non-fermented component is introduced into a pore and/or surface feature of the fermenting component.
